(19) United States
(12) Patent Application Publication
(10) Pub. No.: US 2024/0416020 A1
(43) Pub. Date: Dec. 19, 2024

(54) SPHERICAL CANISTER

(71) Applicant: SPHERICAL CANISTER LLC, Racine, WI (US)

(72) Inventors: Brian T. LEADINGHAM, Pleasant Prarie, WI (US); Patrick C. TETZLAFF, Caledonia, WI (US); Alex J. GRUBER, Hubertus, WI (US); Luke A. WESTHOFF, Kenosha, WI (US); Russ JOHNSON, Libertyville, WI (US); Ken CHUNG, Lindenhurst, IL (US)

(21) Appl. No.: 18/639,533

(22) Filed: Apr. 18, 2024

Related U.S. Application Data
(63) Continuation of application No. 17/243,907, filed on Apr. 29, 2021, now abandoned.
Application No. 17/243,907 is a continuation-in-part of application No. 29/744,288, filed on Jul. 28, 2020, now Pat. No. D925729.

(51) Int. Cl. A61M 1/00; B65D 8/00; B65D 25/56; G01F 23/02

(52) U.S. Cl. CPC A61M 1/60; B65D 11/02; B65D 25/56; G01F 23/02

(57) ABSTRACT

A canister for collection and storage of fluids under vacuum, comprising a spherical container having a first hemispherical component and a second hemispherical component. The first and second components are connected at a canister beltline consisting of multiple flanges, to provide an air-tight and liquid tight seal.

### CROSS-REFERENCE TO RELATED APPLICATIONS

The present Application for Patent is a Continuation of U.S. application Ser. No. 17/243,907, filed Apr. 29, 2021, which is a Continuation in-part of U.S. patent application Ser. No. 29/744,288, filed Jul. 28, 2020, now U.S. Pat. No. D925,729, the entireties of which are incorporated by reference.

### BACKGROUND OF THE INVENTION

The present invention relates to vacuum technology and more specifically, to an apparatus for storage of fluids under vacuum. More specifically, the invention relates to a canister having two hemispherically shaped components whereas a seal joint, or interface is located within the useable collection volume. When the two sides are securely combined, the canister assembly is used for storage of fluids under vacuum.

The use of vacuum technology in a hospital setting has become quite common over the last 100 years. The technology has evolved significantly over time and is utilized for patient care during surgery and in the post-surgical recovery process, as well as to manage respiratory secretions. Medical suction is used not only to clear airways and drain fluids, but also to resuscitate critically ill patients, collect waste, recycle blood from the surgical field, assist in difficult deliveries of newborns, facilitate wound healing, and clear waste gasses and smoke from operating rooms. Over time, such technologies have become much more portable, expanding their use beyond hospitals and into extended care locations such as chronic care facilities, outpatient clinics, physician offices, emergency transportation vehicles and patient homes.

In most applications of vacuum technology, excess fluids are deposited in a surgical suction canister. The prior art for surgical suction canisters provides that they can be reusable, semi-reusable, and disposable. Reusable canisters consist of a rigid plastic base and a plastic lid. They were developed to lower the cost per procedure associated with waste fluid management. Semi-reusable canisters feature disposable liners and lids combined with a reusable outer canister which is not intended to have contact with waste fluid. Disposable suction canisters consist of a base canister and a lid. These are the most common solution used today for the collection and temporary storage of waste fluid.

Reusable fluid collection systems require the complete disposal of waste from the canister after each use. The canisters must also be thoroughly cleaned and disinfected after disposing of the waste. This process of disposal runs the risk of employee exposure to the droplets or aerosols.

Semi-reusable fluid collection systems require clinicians to remove and discard the disposal liner and lid assembly from the rigid outer canister, but the canisters must be thoroughly cleaned and disinfected prior to re-use to avoid risks of cross contamination to the next procedure. This process of disposal runs the risk of employee exposure to the droplets or aerosols.

Disposable canisters provide the benefit of reducing risk of exposure and cross contamination because they are discarded after each use. Disposable canisters, however, are challenged to minimize the amount of plastic used to reduce the cost and plastic material waste, yet they need to be strong enough to withstand required pressures.

While suction canisters have longstanding history of successful use in the health care environment, there are opportunities for improvement in cost and safety.

Prior art disposable suction canister systems have taken the same basic shape and construction for decades. Traditional disposable canisters are created using a canister with a lid. The canister shape is mostly cylindrical and typically injection molded from a plastic resin with high stiffness such as crystal polystyrene or polycarbonate. The lids are generally flat or slightly domed and contain ports for tubing attachment. The lids are typically made from commodity plastics such as ABS, Polystyrene, Polyethylene or Polypropylene. Collected fluid resides within the canister and the lid seals the top of the canister so that vacuum levels from the suction source can be maintained.

In the prior art canister systems, a sealing rib is located on the lid and comes into contact with the canister when the lid is assembled onto the canister; note that the lid to the canister seal interface is always located above the collected fluid volume, hence it is not designed, nor required to be a liquid seal. The sealing rib is typically dimensionally larger than the inner diameter of the canister creating interference between the canister and the sealing rib on the lid. The interference results in localized surface pressure between the sealing rib and the canister. The amount of interference is not arbitrary. Excessive dimensional interference can create difficulties installing the lid on to the canister as the force required to install the lid increases as the amount of dimensional interference increases. Too little interference can result in fluid leaks, preventing proper system functionality. Further, any changes in the structure of the lid or the canister, such as cutouts for latches in the lid, locally alter the structure of the lid creating a discontinuity in the sealing pressure. Latches are commonly used to secure the lid to the canister and are designed to lock the lid onto the base. The discontinuity in pressure about the canister created by the latch is a source of localized deformation resulting in canister air leakage. Additionally, the high levels of lid deformation in prior art canisters also creates changes in the pressure distribution as the lid deforms under vacuum.

In the prior art, a suction canister is under constant vacuum during use with high vacuum settings. One of the primary structural functions of the canister is to withstand the high levels of vacuum without deforming, leaking, or breaking. In terms of structural integrity, there is the issue of lid deformation on existing cylindrical canisters. Lids on existing cylindrical canisters are nearly flat or slightly domed in order to increase lid stiffness. Under high vacuum, traditional lids will often deform significantly, effectively storing mechanical energy while vacuum is applied. If the vacuum is released too quickly when the canister is nearly full, or released without following the specified procedures, the lid can rebound to its original position causing a rapid release of mechanical energy. This rapid lid movement can cause a condition often referred to as canister reflux. During a reflux event, it is possible that waste fluid can spray from the canister or canister ports placing clinicians and equipment at risk of exposure to potentially dangerous fluids.

The inherent risks and costs associated with reusable and semi-reusable canisters provides for a need for disposable rigid suction canisters as the potential for cross contamination is eliminated when compared with reusable and semi-reusable solutions. Disposable canisters also minimize the opportunity for clinicians to come in contact with waste fluid. However, a cost concern arises with disposable canisters in the amount of plastic waste generated.

As such, a need exists for a disposable canister to meet the shortcomings of the prior art.

### SUMMARY OF THE INVENTION

The present invention is a canister for vacuum storage and distribution of fluids. The canister comprises a first hemispherical component and a second hemispherical component that fit together about a beltline in sealable fashion and provide a fluid holding volume for fluid storage.

The canister design addresses the challenges facing traditional disposable canisters by means of applying a liquid tight seal within the useable fluid collection volume.

A first hemispherical component and a second hemispherical component are sealably connected at a beltline to form the spherical shape of the canister with a cavity for storing liquid. The beltline is located below the maximum fluid collection height in the cavity, and within the collection volume of the canister cavity. The collection volume is the volume of the cavity capable of storing a fluid. As a result, fluid in the canister cavity may be in contact with the beltline. Therefore, with liquid in contact with the sealing location of the canister or beltline, a liquid tight and air-tight seal is established and maintained under typical operating conditions. Applicant defines the term “beltline” for the purpose of this invention to be an equatorial seal about a perimeter of the canister positioned at least substantially close to halfway between the poles of the hemisphere components of the canister. If there is a question between the definitions provided in this application for either of “collection volume”, “fluid holding volume”, “collection height” or “beltline” and the common definition of these terms, it is understood that both definitions apply.

Positioning the sealing location between the hemispherical components within the collected volume range is essential as it uniquely provides the opportunity for creating the most efficient design possible, which achieves the necessary strength requirements while minimizing the amount of plastic used all in a design which can be produced, transported, and stored economically.

The spherical canister of the present invention has at least one molded polished surface at the sealing location of the beltline.

In yet another aspect of the present invention, the canister may be comprised of a polymer seal, e.g. a thermoplastic polymer, which provides sealable contact for the canister.

The spherical canister may also include indicia for a measurement of a volume of the fluid within the cavity.

In a further aspect of the invention, the first hemispherical component of the canister has a domed shape to resist mechanical stress or stresses during vacuum storage.

The canister may also include latches or adhesives to contribute to the sealable arrangement between the hemispherical components of the present invention.

Alternatively, the canister may have an ellipsoid shape made of hemispherical components. This shape may further have a third cylindrical component separating the hemispherical components.

These and other features will be described in further detail below.

### BRIEF DESCRIPTION OF THE DRAWINGS

FIG. 1 is a perspective view of a first embodiment of a canister of the invention with hemispherical components of the canister separated.

FIG. 2 is a perspective view of FIG. 1 with the hemispherical components of the canister in connection along a beltline of the canister.

FIG. 3 is a cross-sectional view, taken through line 3-3 of FIG. 2, illustrating the hemispherical components of the canister separated along the beltline of the canister.

FIG. 3A is a close-up perspective cross-sectional view, taken through line 3A-3A of FIG. 2.

FIG. 4 is a cross-sectional view, taken through line 4-4 of FIG. 2, illustrating the hemispherical components of the canister in connection along the beltline of the canister.

FIG. 5 is a close-up cross-sectional view, taken through line 5-5 of FIG. 2, illustrating the hemispherical components of the canister in connection along the beltline of the canister.

FIG. 6 is a close-up focused perspective view of the hemispherical components of FIG. 1, along the beltline of the canister, illustrating a beltline latch of the first hemispherical component of the canister.

FIG. 7 is a cross-sectional view, through line 7-7 of FIG. 6 illustrating the hemispherical components of the canister separated at the beltline latch.

FIG. 8 is a cross-sectional view, taken through line 8-8 of FIG. 6 illustrating the hemispherical components of the canister in close proximity to connection at the beltline latch.

FIG. 9 is a close-up focused cross-sectional view, taken through line 9-9 of FIG. 6, illustrating the hemispherical components of the canister in connection at the beltline latch.

FIG. 10 is a cross-sectional view of a second embodiment of the canister of the invention, illustrating the hemispherical components of the canister separated along the beltline of the canister.

FIG. 10A is a close-up perspective cross-sectional view of the first hemispherical component of the second embodiment of the canister of the invention.

FIG. 11 is a cross-sectional view of a third embodiment of the canister of the invention, illustrating the hemispherical components of the canister separated along the beltline of the canister.

FIG. 11A is a close-up cross-sectional view of the embodiment shown in FIG. 11, illustrating the hemispherical components of the canister in connection at the beltline latch.

FIG. 12 is a cross-sectional view of a fourth embodiment of the canister of the invention, illustrating the hemispherical components of the canister separated along the beltline of the canister.

FIG. 12A is a close-up cross-sectional view of the embodiment in FIG. 12, illustrating the hemispherical components of the canister in connection at the beltline latch.

FIG. 13 is a close-up perspective view of the hemispherical components of the canister shown in FIG. 12.

FIG. 13A is a close-up perspective view the embodiment shown in FIG. 13, illustrating a second embodiment of the beltline latch of the first hemispherical component of the canister in a connected position.

FIG. 14 is a bottom perspective view of the first hemispherical component of a fifth embodiment of the canister.

FIG. 14A is a close-up bottom perspective view of the canister in FIG. 14, illustrating a connection ledge.

FIG. 14B is a close-up cross-sectional view of the canister in FIG. 14, illustrating the hemispherical components of the canister in connection at the beltline latch.

FIG. 15 is a further cross-sectional view of the canister in FIG. 14, illustrating the hemispherical components of the canister separated along the beltline of the canister.

FIG. 15A is a close-up cross-sectional view of another embodiment of the canister of the invention, illustrating the hemispherical components of the canister in connection at the beltline latch.

FIG. 16 is a cross-sectional view, taken through line 16-16 of FIG. 2, illustrating an internal volume of the canister filled with a liquid to a first position above the beltline.

FIG. 17 is a cross-sectional view, taken through line 17-17 of FIG. 2, illustrating the internal volume of the canister filled with the liquid to a second position in close proximity to a top of the canister.

FIG. 18 is a close-up perspective view of the second hemispherical component of the canister, illustrating a second embodiment of indicia.

FIG. 19A is a side view of a seventh embodiment of the canister.

FIG. 19B is a side view of an eighth embodiment of the canister.

FIG. 19C is a side view of a ninth embodiment of the canister.

FIG. 19D is a side view of a tenth embodiment of the canister.

### DESCRIPTION OF THE PREFERRED EMBODIMENT

Although the disclosure hereof is detailed and exact to enable those skilled in the art to practice the invention, the physical embodiments herein disclosed merely exemplify the invention, which may be embodied in other specific structures. While the preferred embodiment has been described, the details may be changed without departing from the invention, which is defined by the claims.

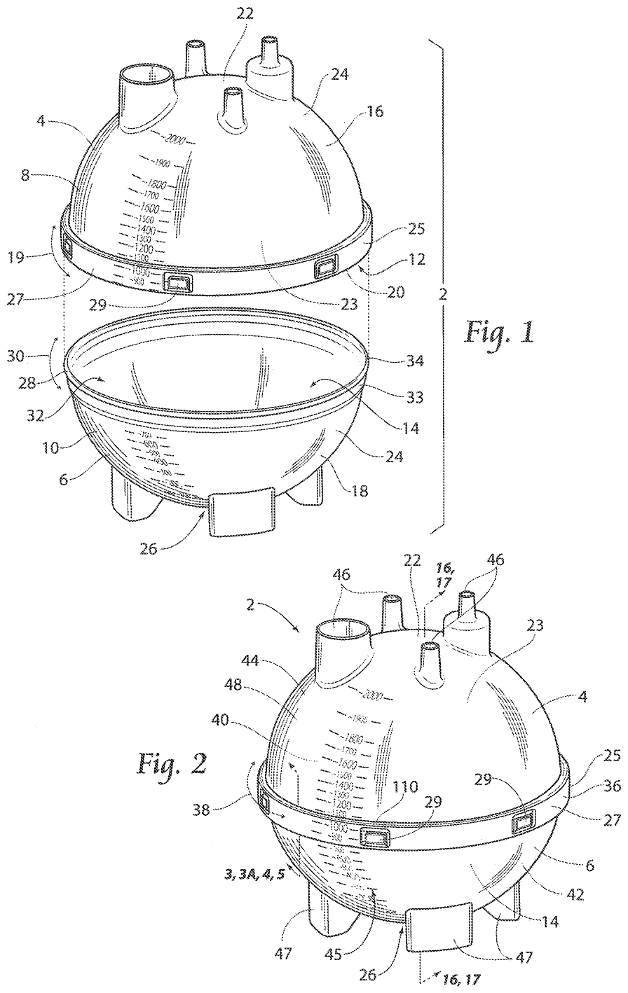

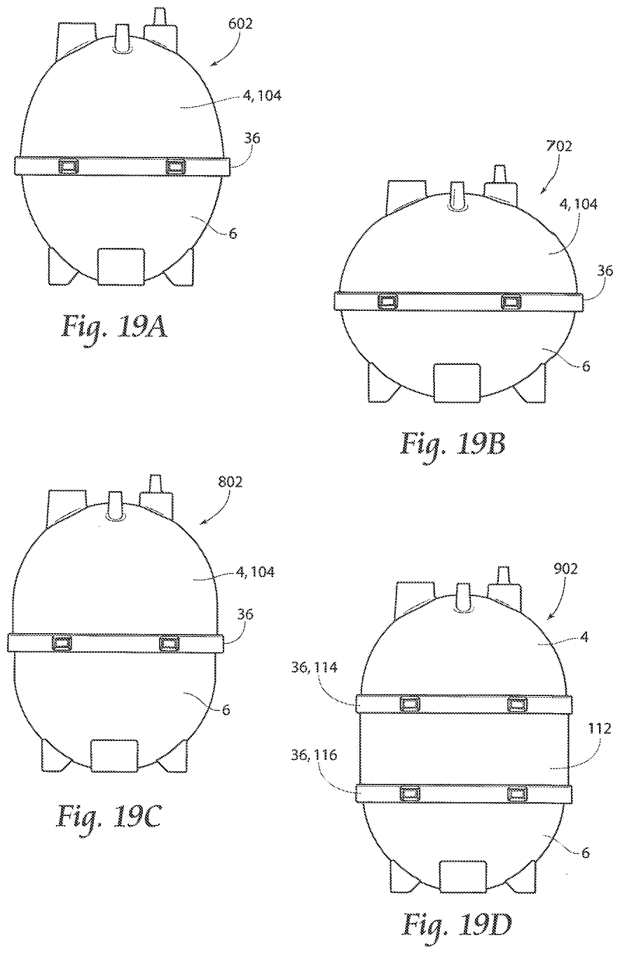

With attention to FIGS. 1 and 2, a first embodiment of a canister 2 of the invention is illustrated. The canister 2 comprises a first hemispherical 4 and a second hemispherical component 6. As will be described below, the hemispherical components are essentially symmetrical of one another and form a spherical shape when joined to one another. Alternatively, the canister may have an ellipsoid shape made of hemispherical components (4,6), sec FIGS. 19A to 19C. This shape may further have a third cylindrical component separating the hemispherical components (4,6), see FIG. 19D.

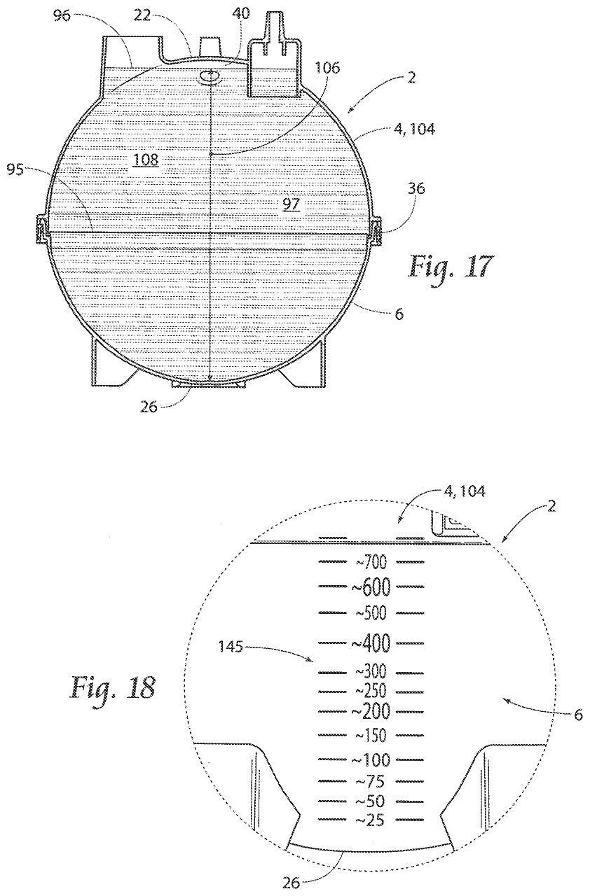

The first hemispherical component 4 and the second hemispherical component 6 are sealably connected at a beltline 36 to form the canister having a cavity 40 for storing liquid. This beltline 36 may be located below the height of the fluid in the cavity, the collection height 106 (see FIGS. 16 and 17), and within the collection volume 108 (sec FIGS. 16 and 17), fluid holding volume, of the canister cavity 40. The collection volume 108 (see FIGS. 16 and 17) is the volume of the cavity 40 which is provided to store a fluid. As a result, fluid in the canister cavity 40 (see FIGS. 4) may be in contact with the beltline 36. Therefore, a liquid tight and air-tight seal will be maintained under typical operating conditions even with a negative pressure applied within the cavity 40 and liquid in contact with the sealing location of the canister 2. The first hemispherical component 4 is defined by a first hemispherical component shell 8 defined by a first hemispherical component outer surface 16. The first hemispherical component 4 has a first hemispherical component pole 22, with the shell 8 extending from the pole 22 in a parabolic shape 24 to a first hemispherical component base 20, thereby defining the first hemispherical component 4. The first hemispherical component base 20 has a first hemispherical component base perimeter 19. The base 20 has a first hemispherical component opening 12. The first hemispherical component opening 12 extends towards the pole 22, forming a first hemispherical component cavity 23. A rim 25 is located at the base 20 around the first hemispherical component base perimeter 19. The rim 25 defines a first hemispherical component beltline 27. Positioned along the first hemispherical component beltline 27 are a plurality, e.g. six, latches 29 to assist in securely attaching the hemispherical components 4 and 6 to one another. Preferably, the latches 29 are positioned equidistant from one another.

Still referring to FIGS. 1 and 2, the second hemispherical component 6 is defined by a second hemispherical component shell 10, further comprising a second hemispherical component outer surface 18. Similar to the shape of the first hemispherical component 4, the second hemispherical component 6 has a second hemispherical component pole 26, with the shell 10 extending from the pole 26 in a parabolic shape 24 to a second hemispherical component base 28, thereby defining the second hemispherical component 6. The second hemispherical component base 28 has a second hemispherical component base perimeter 30. The base 28 has a second hemispherical component opening 32, extending towards the pole 26, forming a second hemispherical component cavity 14. A rim 33 is located at the base 28 around the second hemispherical component base perimeter 30. The rim 33 further has a lip 34.

The second hemispherical component base 28 is positioned in alignment with, and facing, the first hemispherical component base 20. The alignment of the bases (20, 28) is provided by alignment of the first hemispherical component shell 8, at the first hemispherical component base 20, with the second hemispherical component shell 10, at the second hemispherical component base 28.

Still referring to FIGS. 1 and 2, the hemispherical components 4 and 6 are connected along a canister beltline 36. The canister beltline comprises the rim 25 and the lip 34 in communication with one another about the canister perimeter 38. The canister perimeter 38 is substantially equivalent to either or both of the first hemispherical component base perimeter 19 and the second hemispherical component base perimeter 30. The latches 29 also comprise part of the canister beltline 36. Thus, the first hemispherical component cavity 23 and second hemispherical component cavity 14 combine along the beltline 36 to provide for a canister cavity 40, defined by a canister shell 42.

Referring further to FIGS. 1 and 2, the canister shell 42 has an outer surface 44 with indicia 45 extending essentially from pole 22 to pole 26. The indicia 45 provide volume measurements of the amount of fluid within the canister 40, and will be discussed further below on FIG. 17.

The canister 2 also includes one or more ports 46, e.g. four ports, located in close proximity to the first hemispherical component pole 22. The ports 46 extend upward from the first hemispherical component shell 8 and are preferably molded with the shell 8 as a unitary piece.

On the opposite shell 10, there are a plurality of support members 47 located near the second hemispherical component pole 26. The support members 47 provide a means for supporting the canister 2 on a surface. As demonstrated, there are three support members 47 shown, though more or fewer support members could be used. Provided that the support means 47 allow for the canister 2 to rest on a surface, it is understood the arrangement will fall within the scope of the present invention. Alternatively, support members 47 could be eliminated and the canister can rest upon a separate annular shaped or elliptical shaped base structure.

As observed, the canister 2 preferably comprises a spherical shape 48. As such, the canister provides a pressure vessel unique from the prior art. It has been observed, when a stress calculation is performed on the canister 2, the stress in the shells (8,10) of the canister 2 components is ½ of a comparable cylindrical pressure vessel of the prior art. Therefore, the canister 2 of the invention withstands at least the same vacuum pressure of a cylindrical canister of the prior art while requiring the canister shell 42 to be at least half the wall thickness, if not thinner, of a comparable sized cylindrical canister of the prior art. Thus, the canister 2 design provides for reduced manufacturing costs and material costs, and reduces the environmental impact as compared to prior art canisters.

Another advantage to the spherical shape 48, ellipsoidal, or nearly spherical shape, of the canister 2 of the invention is the more efficient shape when comparing the volume of the shape to the surface area of the shape. For example, for a 2000 cubic centimeter suction canister, when comparing the canister 2 of the invention to the prior art cylindrical canister, the canister 2 of the invention occupies 15% less surface floor or surface area when in use or storage as compared to a cylindrical canister of the prior art. In addition, the canister 2 has a reduced spatial occupancy, when in operation and storage, as compared to prior art cylindrical canisters having a substantially equivalent fluid holding volume as that of the canister 2. Spatial occupancy is defined as the volume of space occupied by an object. As with the prior definitions, if there is a question between the definitions provided in this application for “spatial occupancy” and the common definition of this term, it is understood that both definitions apply. The canister 2 design reduces the area occupied by a canister for such use.

In terms of structural integrity the issue of lid deformation, resulting in reflux, on the cylindrical canisters of the prior art is addressed with the present invention. As previously noted, lids on prior art cylindrical canisters are nearly flat or have a very small amount of domed section in order to increase lid stiffness. Under high vacuum traditional lids will often deform significantly, effectively storing mechanical energy while vacuum is applied. When the canister is nearly full, if the vacuum is released too quickly, the lid can rebound to its original position causing a rapid release of mechanical energy. This rapid lid movement can cause a condition often referred to as canister reflux. In contrast, the hemispherical shape of the first hemispherical component 4, provides a lid that is substantially stiffer compared to the prior art cylindrical canisters. The additional stiffness of the present invention makes the design of the invention significantly less prone to reflux, as deformations of the canister 2 are minimal, thus stored energy due to deformation is minimized, ultimately increasing the canister's ability to safely store fluid under vacuum (see FIG. 17).

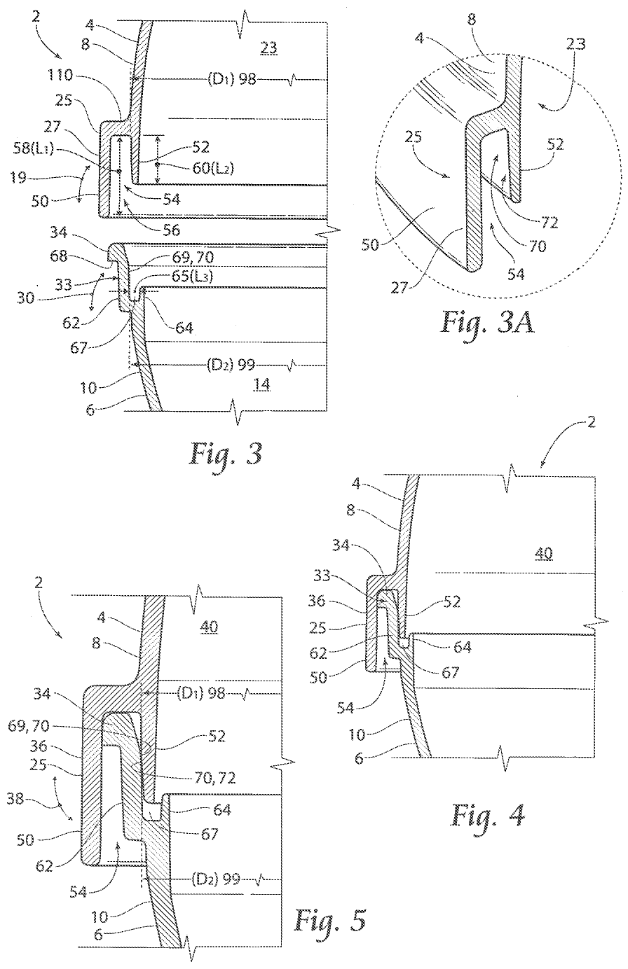

Now referring to FIGS. 3, 3A, 4 and 5, the canister beltline 26 is discussed in further detail. As illustrated in FIG. 3, the hemispherical components (4,6) are separated. The rim 25 comprises two substantially parallel annular ribs, a first hemispherical component outer rib 50 and a first hemispherical component inner rib 52, and a rib shelf 110 connecting the outer rib 50 and the inner rib 52. It is observed the rib shelf 110 contributes to providing a continuous band of positive surface pressure around the entire canister beltline 36 (see FIG. 4) of the canister 2. The rib shelf 110, the outer rib 50 and the inner rib 52 are provided about the first hemispherical component base perimeter 19. The rim 25 has a first hemispherical component channel 54, defined by the positioning of the outer rib 50 and inner rib 52 with respect to the rib shelf 110. The outer rib 50 preferably extends a length L1 (58) and the inner rib preferably extends a length L2 (60). Preferably the length L1 (58) is greater than the length L2 (60). The outer rib 50, and inner rib 52 terminate to provide for a channel opening 56 in the direction of the second hemispherical component 6.

As illustrated in FIG. 3A, the inner rib 52 further provides for an inner rib outer surface 72, facing the outer rib 52 and opposite the first hemispherical component cavity 23 and eventual canister cavity 40, (shown in FIG. 4). During the molding process, the outer surface 72 is molded with a polished finish 70, thereby removing or minimizing manufacturing scratches and imperfections. The polished surface 72 contacts an inner surface 69 of the outer rib 52 of the second hemispherical component 6 to provide for increased sealing properties of the canister 2 as shown in FIGS. 4 and 5. The first hemispherical component inner rib 52 has an outer diameter (D1) 98 defined by the polished outer surface 72 (see FIGS. 3 and 5).

Referring further to FIG. 3, the rim 33 comprises an outer second hemispherical component annular rib 62 and an inner second hemispherical component annular rib 64 extending about the second rib base perimeter 30. The ribs 62 and 64 are substantially parallel with one another, thereby separated by a length L3 (65), and thus define an annular second hemispherical component channel 67 extending about the second hemispherical component base perimeter 30.

The outer rib 33 further provides for an inner surface 69, facing the inner rib 64 and the second hemispherical component cavity 14, and eventual canister cavity 40 (shown in FIG. 4). As noted above, the inner surface 69 also comprises a polished surface 70, to minimize or remove manufacturing scratches and imperfections. The polished surface (69,70) contacts the outer surface 72 of the inner rib of the first hemispherical component to provide for increased scaling properties of the canister 2. As illustrated in FIGS. 3 and 5, the second hemispherical component outer rib 33 has an inner diameter (D2) 99 defined by the polished outer surface (69,70).

Referring further to FIGS. 4 and 5, this provides for an annular lip extension surface 68 extending about the second hemispherical component base perimeter 30 and in a direction opposite that of the second hemispherical component cavity 14, and eventual canister cavity 40.

As illustrated in FIGS. 4 and 5, when the first hemispherical component 4 is combined with the second hemispherical component 6, the outer second hemispherical component annular rib 62 is positioned within the first hemispherical component channel 54. Further, the first hemispherical component inner rib 52 is positioned within, or in substantial proximity to, the second hemispherical component channel 67. The communication of the first hemispherical component 4 and second hemispherical component 6 is along the canister beltline 36 about the canister perimeter 38. In doing so, the ribs (50,52,62,64) are layered in the following orientation from the canister cavity 40: the second hemispherical component inner rib 64, the first hemispherical component inner rib 52, the second hemispherical component outer rib 62 and the first hemispherical component outer rib 50. In this layered orientation, the polished inner surface (69,70) of the outer second hemispherical component annular rib 62 is in substantial contact with the polished outer surface (70,72) of the first hemispherical component inner rib 52 to provide for a liquid tight and air-tight sealing of the canister 2. The liquid tight and air-tight seal are provided because the outer diameter (D1) 98 of the first hemispherical component inner rib 52 is greater than the inner diameter (D2) 99 of the second hemispherical component outer rib 62. This relation causes the inner rib 52 to press against the outer rib 62 and provides for the continuous band of positive surface pressure around the entire canister beltline 36 of the canister 2 between the polished inner surface (69,70) of the outer second hemispherical component annular rib 62 and the polished outer surface (70,72) of the first hemispherical component inner rib 52 is achieved. Further, the shape provides for minimal distortion, thus allowing for the continuous band of positive surface pressure to be achieved and maintained about the beltline 36. As previously noted, the beltline 36, connection location 95 (see 16 and 17), may be located within a collection height 106 (see FIGS. 16 and 17) of the canister volume 40. As a result, fluid in the canister may be in contact with the beltline 36, while a liquid tight and air-tight seal is maintained.

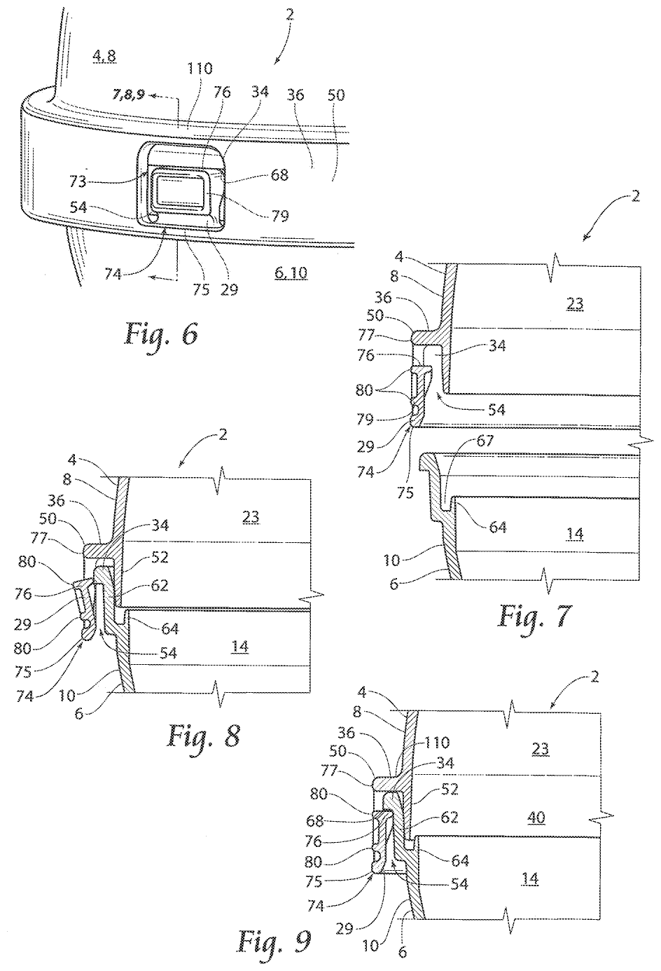

Now referring to FIGS. 6, 7, 8 and 9, communication and connection of the hemispherical components (4,6) at the beltline latch 29 is described. As illustrated in FIG. 6, latch openings 73 along the canister beltline 36 are provided for the beltline latches 29. It is observed, the rib shelf 110 continues through the region of the beltline 36 to define an upper dimension of the latch opening 73. Specifically, each opening 73 comprises a through hole through the first hemispherical component outer rib 50 of the canister beltline 36. The latch 29 is connected to an outer flange, at a first location 74 of the opening 73, at a latch first end 75. The latch 29 extends opposite the first location 74, towards the first hemispherical component pole 22 and terminates at a handle 76 within the opening 73. The handle 76 has an outer lip 79 positioned opposite the first hemispherical component channel 54 and the canister cavity 40. When the canister 2 hemispherical components (4,6) are combined and connected the lip 34 rests upon, and is in communication with, the handle 76.

As illustrated in FIGS. 7, 8 and 9, the interrelationship of the latch 29 and an outer surface 77 of the outer rib 50 gives a visual indication of whether the first hemispherical component 4 and the second hemispherical component 6 are correctly and completely combined to create the canister with the air-tight and liquid tight feature. The outer surface 77 of the outer rib 50 is opposite the channel 54 and is a component of the canister shell outer surface 44 (see FIG. 1). When the hemispherical components (4,6) are separated, the handle outer surface, or surfaces, 80 are substantially planar with respect to the outer surface 77. As illustrated in FIG. 8, the first hemispherical component 4 and second hemispherical component 6 are brought into contact with one another. However, an air-tight and liquid tight seal has not been achieved as illustrated by the position of the handle outer surface, or surfaces, 80 with respect to the outer surface 77. Though the lip 34 of the second hemispherical component outer rib 62 is in contact with the handle 76, they are not in a planar relationship, and the user can see that an airtight relationship has not been achieved.

In contrast, FIG. 9 illustrates the airtight arrangement. The handle outer surfaces 80 are substantially planar with respect to the outer surface 77. The lip extension surface 68 is in contact with and rests upon the handle 76. The polished inner surface (69,70) of the second hemispherical component outer rib 62 and the polished outer surface (70,72) of the first hemispherical component inner rib 52 are in direct contact with one another. Unlike the prior art, each latch 29 is designed as described to provide stiffness to resist excessive deformation under high vacuum loading. Further, the latches 29 further support the maintenance of a continuous band of positive surface pressure around the entire sealing canister beltline 36 of the canister 2, because the rib shelf 110 contributes to providing a continuous band of positive surface pressure at the location of the latch 29. Thus, the latches 29 support maintaining the first hemispherical component 4 in a sealed connection with the second hemispherical component 6.

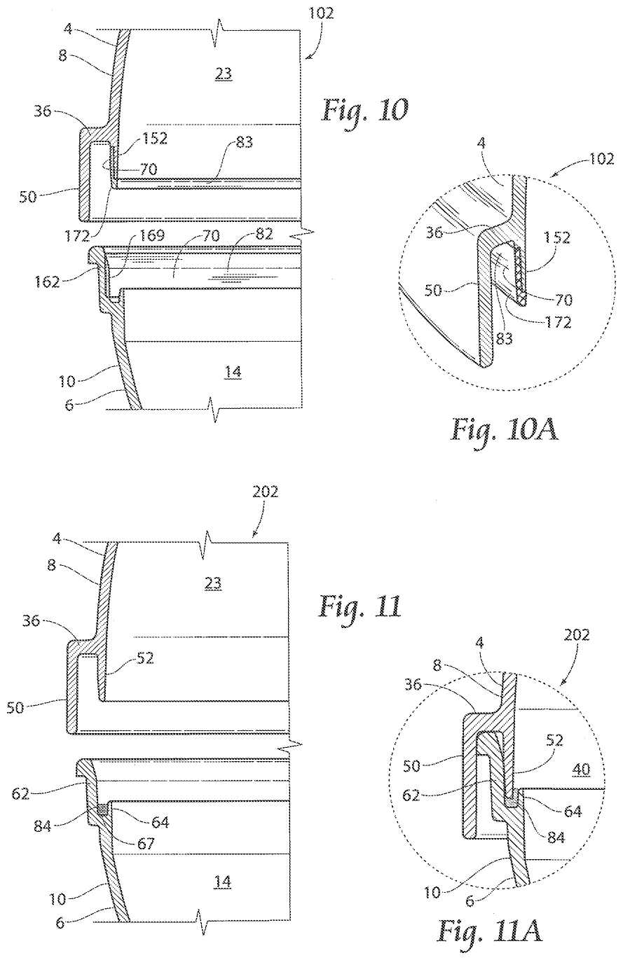

With attention to FIGS. 10 and 10A, a second embodiment of the canister 102 of the invention is illustrated. The canister 102 is similar to canister 2 except that a plastic seal layer is included with canister 102. The second hemispherical component 6 comprises a second hemispherical component outer rib 162. A second hemispherical component polymer section seal 82 is positioned in communication with the rib 162 to provide an inner surface 169. The polymer section 82 is an annular ring in contact with the outer rib 162 along the entirety of the second hemispherical component base perimeter 30. It is understood the inner surface 169 has a polished surface 70.

The first hemispherical component 4 of the canister 102 comprises an inner rib 152. A first hemispherical component polymer section seal 83 is positioned in communication with the inner rib 152 to provide an inner rib outer surface 172. The first hemispherical component polymer section 83 is an annular ring in contact with the inner rib 152 along the entirety of the first hemispherical component base perimeter 19, canister perimeter 38. The outer surface 172 provides for a polished surface 70. Contact between the polished inner surface (169,70) and the polished outer surface (70,172) provides the same sealing and structural properties as described for the contact between the inner surface (69,70) and the outer surface (70,72).

The polymer sections (82, 83) are preferably comprised of a thermoplastic elastomer (TPE). The polymer section 82 is co-molded with the second hemispherical component 6, and the polymer 83 is co-molded with the first hemispherical component 4.

With attention to FIGS. 11 and 11A, a third embodiment of the canister 202 is described. The canister 202 is similar to the canisters 2 and 102, but further has a second hemispherical component channel polymer section seal 84 is positioned in the channel 67 of the second hemispherical component 6. The polymer section 84 is an annular ring in contact with the surfaces of the channel 67 along the entirety of the second hemispherical component base perimeter 30. The polymer sections (82, 83) are preferably comprised of a thermoplastic elastomer (TPE). The polymer section 82 is co-molded with the second hemispherical component 6. As illustrated in FIG. 11A, when the first hemispherical component 4 and second hemispherical component 6 are in contact as previously described, the rib 52 compresses the polymer section 84 along the entirety of the second hemispherical component base perimeter 30, to provide for an additional mode of sealing the canister 202, in addition to the modes previously described.

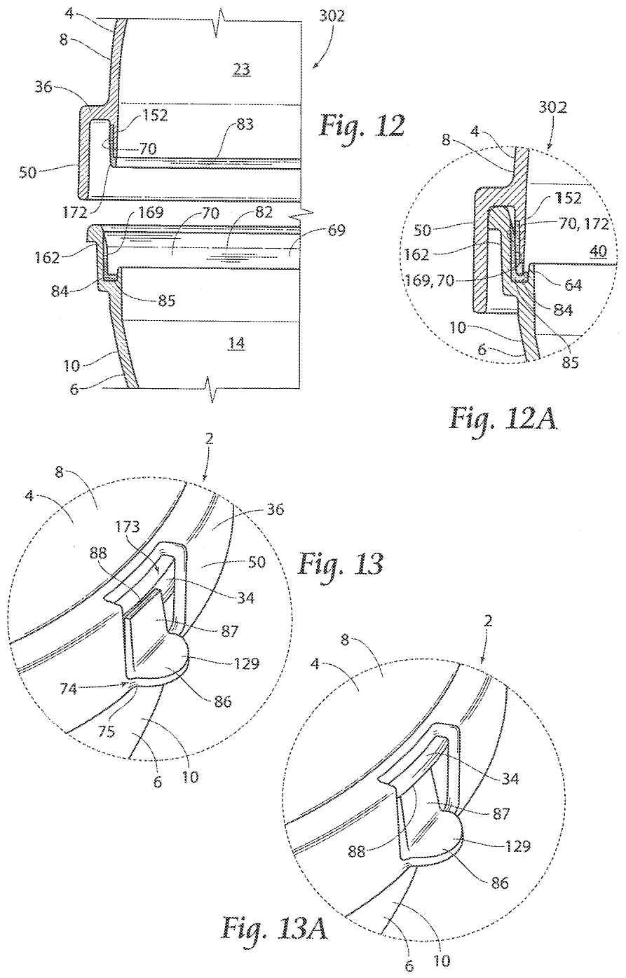

FIGS. 12 and 12A describe a fourth embodiment of the canister 302. The canister 302 incorporates the first hemispherical component 4, with the polymer section 83, of the described with respect to canister 102, above. Further, canister 302 combines the polished outer rib inner surface (169,70) of canister 102 and the second hemispherical component channel polymer section 84 of canister 202 to provide for a second hemispherical component polymer section seal 85. The polymer section 85 is preferably comprised of a thermoplastic elastomer (TPE). The polymer section 85 is co-molded with the second hemispherical component 6. As illustrated in FIG. 12A, it understood the canister 302 provides for the same sealing and structural support properties as described in the previous embodiments.

With attention to FIGS. 13 and 13A, a further beltline latch 129 is illustrated, which can be incorporated into any of the embodiments, noted above. The hemispherical components 4 and 6 of the canister 2 are combined to achieve a seal, as previously described. Along the canister beltline 36 latch openings 173 are provided for the beltline latches 129. Specifically, each opening 173 is provides a through hole through the first hemispherical component outer rib 50 of the canister beltline 36. The latch 129 is connected to the outer flange, at a first location 74 of the opening 173, at a latch first end 75. The latch 129 provides for a manual tab 86 extending from the latch first end 75, and a substantially vertical section 87 extending substantially orthogonal to the manual tab 86. The vertical section 87 terminates with a vertical section top surface 88, which is colored. When the canister hemispherical components (4,6) are in the process of being combined, the lip 34 advances the vertical section 87 pivotably about the latch first end 75. In doing so, the vertical section top surface 88 is exposed to reveal the colored shading. The colored shading indicates the canister 2 is not sealed. As illustrated in FIG. 13A, when the canister 2 hemispherical components (4,6) are combined and connected to provide for a sealed canister 2, the lip 34 rests upon, and is in communication with, the vertical section top surface 88. Therefore, the color shading on the vertical section top surface 88 is not visible, thereby verifying the hemispherical components (4,6) are combined and connected to provide a properly sealed canister 2 about the entirety of the beltline. Each latch is designed to provide stiffness to resist excessive deformation under high vacuum loading.

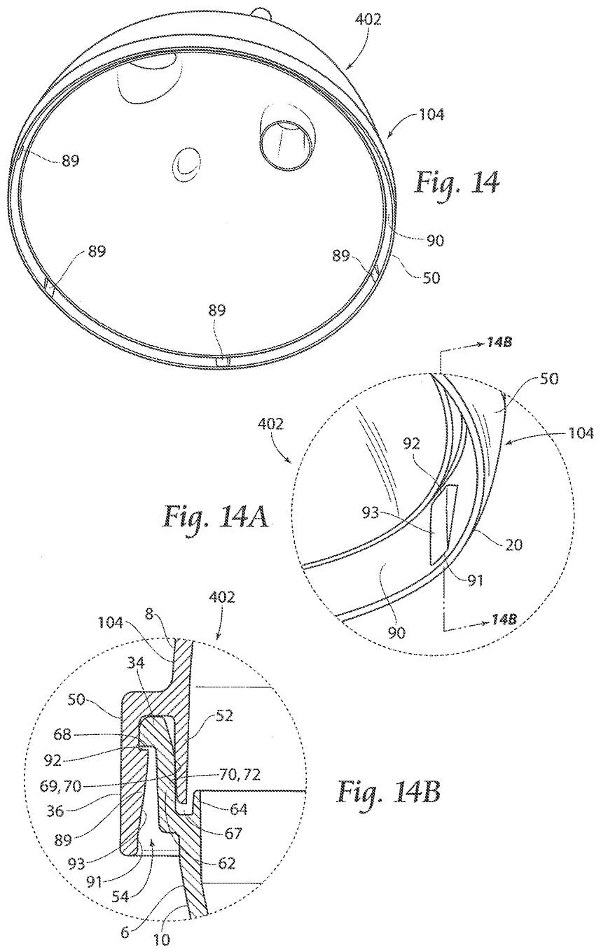

With attention to FIGS. 14, 14A and 14B, a fifth embodiment of the canister 402 is illustrated. The canister 502 incorporates the second hemispherical component 6 as previously described, but incorporates a first hemispherical component 104. The first hemispherical component 104 differs from the previously described first hemispherical component 4 in that internal latch mechanisms 89 are substituted for the latches (29,129) on the first hemispherical component 4, as previously described. The latch mechanisms 89 are molded and positioned on an interior wall 90 of the first hemispherical component outer rib 50. The interior latch mechanisms 89 comprise a wedge configuration with the angled surface 93 of the wedge providing a tapered section 91 in close proximity to the first hemispherical component base 20 and a substantially horizontal surface 92, wherein the tampered section 91 and the substantially horizontal surface 92 are separated by the angled surface 93. The canister 402 has at least one internal latch mechanism 89, but preferably multiple latch mechanisms 89, e.g. six. As illustrated in FIG. 14B, the sealing properties and structural properties are as described in the first embodiment of the canister 2. The lip extension surface 68 of the lip 34 of the second hemispherical component outer rib 62 rests upon the horizontal surface 92 when the hemispherical components (104,6) of the canister 402 are combined to provide for a sealed and structurally sound canister as previously described. The internal latch 89 provides for a mechanism to retain the hemispherical components in position. Each latch is designed to provide stiffness to resist excessive deformation under high vacuum loading.

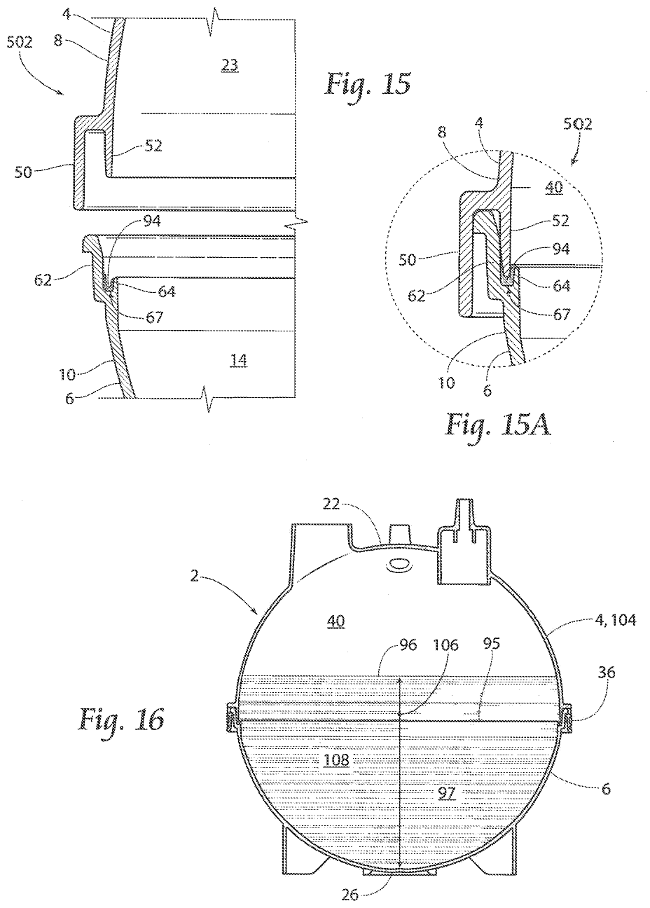

With attention to FIGS. 15 and 15A, a sixth embodiment of the canister 502 is illustrated. In the second hemispherical component 6, a second hemispherical component channel adhesive seal 94 is positioned in the channel 67 of the second hemispherical component 6. The adhesive 94 is an annular ring in contact with the surfaces of the channel 67 along the entirety of the second hemispherical component base perimeter 30. As illustrated in FIG. 15A, when the first hemispherical component 4 and second hemispherical component 6 are in contact as previously described, the rib 52 compresses the adhesive 94 along the entirety of the second hemispherical component base perimeter 30, resulting in the hemispherical components forming a unitary one piece construction. This provides for additional structural and sealing for the canister 502.

As discussed, the layered orientation of the ribs (50,52,62,64,152,162) structurally provides for the first hemispherical component inner rib (52,152) to sealably contact the second hemispherical component outer annular rib (62,162), as well as a polymer section 84 or adhesive 94 in the channel 67. Further, the latch (29,129,89) construction provides stiffness to resist excessive deformation under high vacuum loading. The combination of the rib orientation and the latch construction provide for a continuous positive sealing pressure about the beltline 36 (see FIG. 16). With respect to the latches (29,129,89), the sealed contact between the canisters is ensured through audible clicks of the latches (29,129), color indications as seen on the latches 129 (see FIG. 13), and tactile indication as seen with the comparison of surfaces 77 and 80 on for the latch (see FIG. 9). In the tactile indication the canister beltline 36 has a smooth contour.

As noted above, FIGS. 16 and 17 demonstrate the sealing properties of the present invention. A connection location, line, 95 of the hemispherical components (4,104,6) is illustrated. Unlike the prior art canisters, where the connection location between the lid and the liquid well of the canister is located above the fluid level line of the liquid, the connection location 95 is positioned along the canister beltline 36, which is the connection location for the hemispherical components (4,104,6). The canister beltline 36 is positioned approximately equidistant between the first hemispherical component pole 22 and the second hemispherical component pole 26. Thus, unlike the prior art, the canister beltline 36 is not positioned in close proximity to the first hemispherical component pole 22. As a result, fluid 97 can fill the cavity 40 such that the fluid level line 96 is above the beltline 36, and connection location 95. It is observed the beltline 36 is located within the collection height 106 of the collection volume 108 of the canister volume 40. The sealing properties of the canister, between the hemispherical components (4,104,6) provides for an air-tight and liquid tight seal allowing the fluid level line 96 to be above the connection location 95 between the first hemispherical component (4,104) and the second hemispherical component 6. The spherical, ellipsoidal, or nearly spherical design, design of the present invention maintains a nearly uniform pressure distribution which exceeds the pressure differential due to vacuum, combined with the static pressure due to the height of the waste fluid, in order to maintain a leak free or airtight seal without allowing for visible air bubbles to enter at the seal.

As illustrated in FIG. 17, unlike the prior art, where fluid levels reaching close to a lid posed the risk of a reflux event when pressure was released from the canister, with the design of the canister 2 the fluid level line 96 of the fluid 97 can be very near the top of the canister 2 without a risk of a reflux event. As previously described, the canister 2 structurally resists deformation during a vacuum process. Therefore, when a vacuum is applied to the canister 2, the canister 2 does not store sufficient mechanical energy from deformation of the canister, thereby preventing a reflux event when the vacuum is released.

With attention to FIG. 18, a second embodiment of indicia 145 is illustrated on the second hemispherical component 6. The indicia provide smaller increments at lower volumes. Thus, the second embodiment of indicia 145 provides for accurate measurements of low volumes of liquid (97, see FIGS. 16 and 17) such that the fluid level line (96, see FIGS. 16 and 17) is in close proximity to the second hemispherical component pole 26. This second embodiment of indicia 145 provides for accurate measurement of low, reduced, volumes where such accuracy at low volumes is required, e.g. 300 milliliters and lower volumes. The domed shape of the sphere, alternatively the ellipsoidal shape, provides for an increased distance between low volume indicia 145 as compared to low volume indicia of the cylindrical shaped prior art canisters having the same or similar fluid holding volume as that of the canister 2. This increased distance provides for the more accurate measurements of low fluid volumes, a more precise fluid collection reading, within the canister 2 as compared to fluid collection readings of the prior art, where the prior art has a substantially similar fluid holding volume as compared to the canister 2. Alternatively, dependent upon the orientation of the hemispherical components, the indicia may extend parallel to the beltline.

With attention to FIGS. 19A to 19C, the canister (602,702,802) may have an ellipsoid shape made of the hemispherical components (4,104,6) connected at the beltline 36 as previously described. As illustrated in FIG. 19D, the canister 902 may have an elongated ellipsoid shape made of the hemispherical components (4,104,6) with a cylinder component 112 separating the hemispherical components (4,104,6). The first hemisphere component (4,104) is connected to the cylinder section along a first beltline (36,114). Opposite the first beltline, the section cylinder is connected to the second hemispherical component 6 along a second beltline (36,116). The first beltline and the second beltline comprise the properties of the beltline 36 as previously described.

As demonstrated above, the present invention provides an improved device for storing fluids. The invention provides a durable, fluid tight container that minimizes the potential for fluid spills in a cost-effective and efficient manner. As a result, whether absent a vacuum, under low vacuum or under high vacuum, the canister 2 provides a continuous seal about the canister beltline 36 ensuring safe and efficient storage and disposal of fluids. It should be noted that the various features of the various embodiments could be combined and fall within the scope of the present invention.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

We claim:

1. A spherical canister for vacuum collection and storage of medical fluids, the spherical canister comprising:
a first hemisphere and a second hemisphere;
the first hemisphere including a first base;
the second hemisphere including a second base; and
a first annular rib in scalable contact with a second annular rib about a canister beltline, thereby forming a medical fluid holding volume.
